(19) United States
(12) Patent Application Publication
(10) Pub. No.: US 2019/0338256 A1
(43) Pub. Date: Nov. 7, 2019

(54) REDOX BALANCING IN YEAST

(71) Applicant: DANISCO US INC., Palo Alto, CA (US)

(72) Inventors: Andrei Miasnikov, Union City, CA (US); Barbara Urszula Kozak, Utrecht (NL)

(21) Appl. No.: 16/348,018

(22) PCT Filed: Apr. 24, 2017
(86) PCT No.: PCT/US17/29106

§ 371 Date: May 7, 2019

Related U.S. Application Data
(60) Provisional application No. 62/328,800, filed on Apr. 28, 2016.

(51) Int. Cl. C12N 9/02; C12N 9/88; C12N 9/10; C12P 7/06

(52) U.S. Cl. CPC C12N 9/0008; C12Y 102/01003; C12Y 401/02022; C12P 7/06; C12Y 203/01008; C12N 9/1029; C12Y 203/01054; C12N 9/88

(57) ABSTRACT

Described are composition and methods relate to reducing the redox imbalance in anaerobically growing yeast with attenuated glycerol production by re-engineering the pathway for Ac-CoA biosynthesis.

### CROSS REFERENCE TO RELATED APPLICATIONS

This application claims benefit of priority from US provisional application U.S. Ser. No. 62/328,800, filed 28 Apr. 2016, which is incorporated by reference in its entirety.

### TECHNICAL FIELD

The present composition and methods relate to reducing the redox imbalance in anaerobically growing yeast with attenuated glycerol production by re-engineering the pathway for Ac-CoA biosynthesis. The resulting pathway-modified yeast is useful for producing ethanol from carbohydrate-containing substrates.

### BACKGROUND

In industrial ethanol and biochemical production using yeast to ferment carbohydrate-containing hydrolysates, glycerol is a major low value by-product. Reducing glycerol production and channeling additional carbon into ethanol or other valuable biochemical is, therefore, economically attractive.

One solution to improve ethanol yield during yeast fermentations is to inactivate genes encoding the enzymes that control glycerol formation. Such genes are well known, e.g., GPD1 and GDP2, which encode two glycerophosphate dehydrogenases that convert dihydroxyacetone-phosphate (a glycolytic intermediate) into glycerophosphate, which is subsequently dephosphorylated into glycerol. Yeast strains carrying deletions in these two genes have been described (see, e.g., Bjorkqvist, S. et al. (1997) Appl. Environ. Microbiol., 63:128-32 and Ansell, R. et al. (1997) EMBO J. 16:2179-87). However, such strains are not suitable as industrial ethanologens or biochemical producers because they lack the ability to grow under anaerobic conditions. The underlying physiological reason for the inability of so-called “glycerol-free” yeast strain to grow anaerobically is so-called redox-imbalance.

Although fermentation of glucose into ethanol via the glycolytic pathway is redox-balanced, some of the glycolytic intermediates between glyceraldehyde 3-phosphate (the intermediate oxidized by a NAD⁺-dependent dehydrogenase) and acetaldehyde (the intermediate reduced by a NADH-dependent dehydrogenase) are withdrawn for biosynthetic needs (e.g., building up biomass). Withdrawal of such intermediates results in the production of excessive amounts of NADH and consequent redox imbalance. Yeast generally mitigates the problem of redox imbalance by re-oxidizing the excessive NADH with glycerophosphate dehydrogenase (encoded in yeast by the genes GPD1 and GPD2).

Attempts to solve the problem of redox imbalance in yeast while reducing glycerol formation have been described. For example, a soluble H₂O-forming NADH oxidase has been used to lower the NADH/NAD⁺ ratio in micro-aerobically grown yeast (Heux, S et al. (2006) Metab. Eng. 8:303-14). While conceptually attractive, this approach cannot be applied to yeast grown under completely anaerobic conditions of typical industrial yeast fermentation. Another approach is based on substituting both osmoprotective and redox-balancing functionality of glycerol with alternative molecules such as sugar alcohols (see, e.g., Shen B. et al. (1999) Plant Physiol. 121:45-52).

The main drawback of all the above approaches is that the amount of carbon diverted from the production of ethanol or other valuable biochemicals into the alternative pathway is about the same or higher than the amount that wild-type yeast divert into glycerol production, thereby defeating the original objective.

### SUMMARY

The present composition and methods relate to reducing the redox imbalance in anaerobically growing yeast with attenuated glycerol production by re-engineering the pathway for Ac-CoA biosynthesis. Aspects and embodiments of the compositions and methods are described in the following, independently-numbered paragraphs.

1. In one aspect, modified yeast cells are provided, comprising: an attenuated native biosynthetic pathway for making Ac-CoA, which native pathway contributes to redox cofactor imbalance in the cells under anaerobic conditions; introduction of an artificial alternative pathway for making Ac-CoA, which artificial pathway does not contribute to a redox cofactor imbalance in the cells under anaerobic conditions compared to the native biosynthetic pathway; and attenuation of the glycerol biosynthesis pathway; wherein the modified yeast cells demonstrate increased ethanol production using a carbohydrate substrate compared to a comparable yeast cells lacking the modifications.

2. In some embodiments of the modified yeast cells of paragraph 1, attenuation of the native Ac-CoA pathway is achieved by reducing aldehyde dehydrogenase activity.

3. In some embodiments of the modified yeast cells of paragraph 1 or 2, attenuation of the native Ac-CoA pathway is achieved by reducing the expression of one or more of the native genes encoding aldehyde dehydrogenase (ALD2, ALD3, ALD4, ALD5 or ALD6).

4. In some embodiments of the modified yeast cells of any of paragraphs 1-3, the artificial alternative pathway for making Ac-CoA is the result of introducing exogenous phosphoketolase activity and exogenous phosphotransacetylase activity.

5. In some embodiments of the modified yeast cells of any of paragraphs 1-4, the artificial alternative pathway for making Ac-CoA is the result of introducing a heterologous phosphoketolase gene and a heterologous phosphotransacetylase gene.

6. In some embodiments of the modified yeast cells of any of paragraphs 1-5, attenuation of the glycerol biosynthesis pathway is the disruption or modification of GDP1, GDP2, GPP1 and/or GPP2.

7. In some embodiments of the modified yeast cells of any of the preceding paragraphs, the cells further comprise increased acetyl-CoA synthase activity.

8. The modified yeast cells of any of the preceding paragraphs, wherein the cells further comprise a heterologous gene encoding a polypeptide having acetyl-CoA synthase activity or an overexpressed endogenous gene encoding a polypeptide having acetyl-CoA synthase activity.

9. In some embodiments of the modified yeast cells of any of the preceding paragraphs, the cells lack a heterologous gene encoding a protein with NAD⁺-dependent acetylating acetaldehyde dehydrogenase activity or have reduced NAD⁺-dependent acetylating acetaldehyde dehydrogenase activity.

10. In some embodiments of the modified yeast cells of any of the preceding paragraphs, the cells lack a heterologous gene encoding a pyruvate-formate lyase or have reduced pyruvate-formate lyase activity.

11. In some embodiments of the modified yeast cells of any of the preceding paragraphs, the modified yeast cells demonstrate at least 1%, at least 2%, at least 4%, at least 6%, at least 8% or even at least 10% increased ethanol production using a carbohydrate substrate compared to a comparable yeast cells lacking the modifications.

12. In another aspect, a method for increasing the production of ethanol by yeast cells grown on a carbohydrate substrate is provided, comprising: attenuating the yeast cell native biosynthetic pathway for making Ac-CoA, which native pathway contributes to redox cofactor imbalance in the yeast cells under anaerobic conditions; introducing into the yeast cells an artificial alternative pathway for making Ac-CoA, which artificial pathway does not contribute to a redox cofactor imbalance in the yeast cells under anaerobic conditions compared to the native pathway; and attenuating the glycerol biosynthesis pathway in the yeast cells; wherein the modified yeast cells demonstrate increased ethanol production using a carbohydrate substrate compared to a comparable yeast cells lacking the modifications.

13. In some embodiments of the method of paragraph 12, attenuating the native Ac-CoA pathway is performed by reducing aldehyde dehydrogenase activity.

14. In some embodiments of the method of paragraph 12 or 13, attenuating the native Ac-CoA pathway is performed by disrupting one or more native aldehyde dehydrogenase genes.

15. In some embodiments of the method of any of paragraphs 12-14, the artificial alternative pathway for making Ac-CoA results from introducing exogenous phosphoketolase activity and exogenous phosphotransacetylase activity.

16. In some embodiments of the method of any of paragraphs 12-15, the artificial alternative pathway for making Ac-CoA is the result of introducing a heterologous phosphoketolase gene and a heterologous phosphotransacetylase gene.

17. In some embodiments of the method of any of paragraphs 12-16, attenuating the glycerol biosynthesis pathway is performed by disrupting or modifying GDP1, GDP2, GPP1 and/or GPP2.

18. Some embodiments of the method of any of paragraphs 12-17 further comprise increasing acetyl-CoA synthase activity.

19. Some embodiments of the method of any of paragraphs 12-18 further comprise introducing into the cell a heterologous gene encoding a polypeptide having acetyl-CoA synthase activity or overexpressing an endogenous gene encoding a polypeptide having acetyl-CoA synthase activity.

20. In some embodiments of the method of any of paragraphs 12-19, the cells lack a heterologous gene encoding a protein with NAD⁺-dependent acetylating acetaldehyde dehydrogenase activity or have reduced NAD⁺-dependent acetylating acetaldehyde dehydrogenase activity.

21. In some embodiments of the method of any of paragraphs 12-20, the cells lack a heterologous gene encoding a pyruvate-formate lyase or have reduced pyruvate-formate lyase activity.

22. In some embodiments of the method of any of paragraphs 12-21, the modified yeast cells demonstrate at least 1%, at least 2%, at least 4%, at least 6%, at least 8% or even at least 10% increased ethanol production using a carbohydrate substrate compared to a comparable yeast cells lacking the modifications.

23. In another aspect, modified yeast cells produced by the method of any of paragraphs 12-22 are provided.

24. In another aspect, a method for increasing the production of ethanol by yeast cells grown on a carbohydrate substrate is provided, comprising: incubating a carbohydrate substrate in the presence of the modified yeast cells of any of paragraphs 1-11 or 23 or in the presence of modified yeast cells produced by the method of any of paragraphs 12-22, wherein the modified yeast cells demonstrate at least 1%, at least 2%, at least 4%, at least 6%, at least 8% or even at least 10% increased ethanol production using a carbohydrate substrate compared to a comparable yeast cells lacking the modifications.

25. In another aspect, ethanol produced by the method of paragraph 24 or 28 is provided.

26. In another aspect, modified yeast cells, comprising the acetyl-CoA synthase from Methanosaeta concilii (WP_013718460) or an enzyme having at least 85%, at least 90%, at least 92%, at least 95%, at least 97%, at least 98% and even at least 99% amino acid sequence identity the acetyl-CoA synthase from Methanosaeta concilii, are provided.

27. In another aspect, a method for converting acetate to Ac-CoA comprising introducing into the cell a heterologous gene encoding a polypeptide having acetyl-CoA synthase activity, wherein the gene is derived from the acetyl-CoA synthase from Methanosaeta concilii (WP_013718460) or an enzyme having at least 85%, at least 90%, at least 92%, at least 95%, at least 97%, at least 98% and even at least 99% amino acid sequence identity the acetyl-CoA synthase from Methanosaeta concilii, is provided.

28. In another aspect, a method of paragraph 27 used in combination with the method of any of paragraphs 12-22 or 24 is provided.

### BRIEF DESCRIPTION OF THE DRAWINGS

FIG. 1 is an image showing the growth of two isolates of the FG ura3-derived strains carrying deletions in ald4, ald5 and ald6 streaked on SC ura+ (2% glucose, 0.67% yeast nitrogen base w/o amino acids, 200 mg/l uridine) plates (left) and the same medium supplemented with 1 g/l potassium acetate (right). Both plates were incubated for about 3 days at 30° C.

FIG. 2 is a diagram depicting a map of plasmid pPATH6(FBA1).

FIG. 3 is a graph showing a glucose consumption time course experiment measuring glucose consumption (g/L) by engineered strains ZG1::pPATH6(FBA1) and ZZ::pPATH6(FBA1) as well as reference (and comparative wild-type) strain FERMAX™ Gold.

FIG. 4 is a graph showing ethanol production (g/L) by ZG1::pPATH6(FBA1), ZZ::pPATH6(FBA1) and FERMAX™ Gold.

FIG. 5 is a graph showing end of fermentation titers of glycerol (g/L) and ethanol (g/L) in cultures of ZG1::pPATH6(FBA1), ZZ::pPATH6(FBA1) and FERMAX™ Gold.

### DETAILED DESCRIPTION

### I. Introduction

The present composition and methods relate to increasing the yield of ethanol from yeast fermentation under anaerobic conditions by a strategy that is different from those previously described. Rather than trying to alleviate the redox imbalance that results from reducing or eliminating the production of glycerol as an electron sink, the present strategy is to modifying yeast metabolism to reduce or eliminate the root cause of this redox imbalance. One major pathway contributing to creation of redox imbalance in anaerobically fermenting yeast is the Ac-CoA biosynthesis pathway, which is used by yeast for making the essential Ac-CoA precursor under anaerobic conditions. In anaerobically growing yeast, Ac-CoA synthesis involves oxidation of acetaldehyde by a NADP⁺-dependent dehydrogenase into acetic acid, which is subsequently converted into Ac-CoA by acetyl-CoA synthetase. The acetaldehyde diverted into this pathway cannot be used for NADH-dependent reduction into ethanol catalyzed by alcohol dehydrogenase. Since a molecule of NAD⁺ is spent on oxidizing glyceraldehyde 3-phosphate upstream in the glycolytic pathway, diversion of acetaldehyde into acetate is a contributor to the redox imbalance.

Surprisingly, we have found that attenuation of the native Ac-CoA pathway at the aldehyde dehydrogenase step, and providing yeast with an alternative Ac-CoA biosynthetic pathway based on phosphoketolase, is sufficient to eliminate much of the anaerobic growth incompetence of glycerol-free yeast strains. Attenuation of the native Ac-CoA pathway reduces the accumulation NADH because the acetaledyde that would normally be withdrawn into this pathway in wild-type yeast becomes available as a substrate for alcohol dehydrogenase, which recycles NADH into its oxidized form, NAD⁺, while the introduction of a phosphoketolase-based pathway restores the production of Ac-CoA from five or six-carbon sugar-phosphate precursors in a redox-independent manner.

The yield of ethanol is significantly improved using such engineered strains. Yields are also improved in strains with alternative Ac-CoA biosynthetic pathway that are modified to have only partial attenuation of glycerol-forming pathway; therefore, complete elimination of the wild-type glycerol biosynthetic pathway is not required to realize a benefit in ethanol production.

### II. Definitions

Prior to describing the present strains and methods in detail, the following terms are defined for clarity. Terms not defined should be accorded their ordinary meanings as used in the relevant art.

As used herein, the term “gene” is synonymous with the term “allele” in referring to a nucleic acid that encodes and directs the expression of a protein or RNA.

As used herein, the terms “wild-type” and “native” are used interchangeably and refer to genes, proteins, strains, and biochemical pathways found in nature.

As used herein, “deletion of a gene,” refers to its removal from the genome of a host cell. Deletion may be complete, meaning the an entire gene (i.e., at least the entire coding sequences are removed) or partial, meaning that only a portion of the coding sequences or regulatory sequences are removed but which prevent the production of a functional gene product.

As used herein, “attenuation of a pathway” or “attenuation of the flux through a pathway” i.e., a biochemical pathway, refers broadly to any genetic or chemical manipulation that reduces or completely stops the flux of biochemical substrates or intermediates through a metabolic pathway. Attenuation of a pathway may be achieved by a variety of well-known methods. Such methods include but are not limited to: complete or partial deletion of one or more genes, replacing wild-type alleles of these genes with mutant forms encoding enzymes with reduced catalytic activity or increased Km values, modifying the promoters or other regulatory elements that control the expression of one or more genes, engineering the enzymes or the mRNA encoding these enzymes for a decreased stability, misdirecting enzymes to cellular compartments where they are less likely to interact with substrate and intermediates, the use of interfering RNA, and the like.

As used herein, “disruption of a gene” refers broadly to any genetic manipulation that substantially prevents a cell from producing a functional gene product. Exemplary methods of gene disruption include complete or partial deletion of a gene and making mutations in coding or regulatory sequences.

As used herein, the terms “genetic manipulation” and “genetic alteration” are used interchangeably and refer to the alteration/change of a nucleic acid sequence. The alteration can included but is not limited to a substitution, deletion, insertion or chemical modification of at least one nucleic acid in the nucleic acid sequence.

As used herein, the term “substantially prevents the functioning of the pathway,” means that the activity of the pathway, as measured by the consumption or production of a product indicative of the functioning of the pathway, is reduced at least 50%, at least 60%, at least 70%, at least 80%, at least 90%, at least 95%, %, or even undetectable, compared to the corresponding pathway in unmodified (i.e., wild-type) cells.

As used herein, the phrase “substantially prevents a cell from producing a functional gene product,” or similar phrases, means that the activity of a specified gene product, as measured by the consumption or production of a product indicative of the functioning of the gene product, is reduced at least 50%, at least 60%, at least 70%, at least 80%, at least 90%, at least 95%, or even undetectable, compared to levels of the corresponding gene product in unmodified (i.e., wild-type) cells. As used herein, the phrase “substantially free of an activity,” substantially prevent or similar phrases, means that a specified activity is reduced at least 50%, at least 60%, at least 70%, at least 80%, at least 90%, at least 95%, or even undetectable, compared to the corresponding activity in unmodified (i.e., wild-type) cells.

As used herein, “aerobic fermentation” refers to growth in the presence of oxygen.

As used herein, “anaerobic fermentation” refers to growth in the absence of oxygen.

As used herein, the terms “polypeptide” and “protein” (and/or their respective plural forms) are used interchangeably to refer to polymers of any length comprising amino acid residues linked by peptide bonds. The conventional one-letter or three-letter codes for amino acid residues are used herein. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity.

As used herein, the term “percent amino acid sequence identity,” or similar, means that a particular sequence has at least a certain percentage of amino acid residues identical to those in a specified reference sequence, when aligned using the CLUSTAL W algorithm with default parameters. See Thompson et al. (1994) Nucleic Acids Res. 22:4673-80. Default parameters for the CLUSTAL W algorithm are:
Gap opening penalty: 10.0
Gap extension penalty: 0.05
Protein weight matrix: BLOSUM series
DNA weight matrix: IUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:
EC enzyme commission
kDa kiloDalton
kb kilobase
MW molecular weight
w/v weight/volume
w/w weight/weight
v/v volume/volume
wt % weight percent
° C. degrees Centigrade
H₂O water
H₂O₂ hydrogen peroxide
dH₂O or DI deionized water
dIH₂O deionized water, Milli-Q filtration
g or gm gram
μg microgram
mg milligram
kg kilogram
lb pound
μL and μl microliter
mL and ml milliliter
mm millimeter
μm micrometer
mol mole
mmol millimole
M molar
mM millimolar
μM micromolar
nm nanometer
U unit
ppm parts per million
sec and ″ second
min and ′ minute
hr and h hour
EtOH ethanol
eq. equivalent
PCR polymerase chain reaction
DNA deoxyribonucleic acid
A relating to a deletion
bp base pairs

### III. Embodiments

A. Overview of Modified Yeast

The present compositions and methods relate to increasing the yield of ethanol from yeast fermentation under anaerobic conditions by modifying yeast metabolism to reduce glycerol production and reduce and or eliminate the redox imbalance. The present compositions and methods include yeast strains that have an attenuated native Ac-CoA pathway, e.g., at the aldehyde dehydrogenase step, and which are provided with an alternative Ac-CoA biosynthetic pathway based on phosphoketolase. This combination of genetic manipulations is shown to be sufficient to eliminate much of the anaerobic growth incompetence of glycerol-free yeast strains. Attenuation of the native Ac-CoA pathway reduces the accumulation NADH because the acetaledyde that would normally be withdrawn into this pathway in wild-type yeast becomes available as a substrate for alcohol dehydrogenase, which recycles NADH into its oxidized form, NAD⁺, while the introduction of a phosphoketolase and phosphotransacetylase restores the production of Ac-CoA from five or six-carbon sugar-phosphate precursors in a redox-independent manner. The key features of the present compositions and methods are described in more detail, below:

B. Attenuation of the Native Biosynthetic Pathway in Yeast for Making Ac-CoA

A first feature of the present compositions and methods is attenuation of the native biosynthetic pathway in yeast for making Ac-CoA, which contributes to redox cofactor imbalance in the cells under anaerobic conditions and thus requires glycerol production for restoring the redox balance. In some embodiments, the compositions and methods involve disruption of one, several or all the native genes (e.g., ALD2 ALD3 ALD4 ALD5 and ALD6) encoding aldehyde dehydrogenase (EC 1.2.1.3). The native yeast Ac-CoA pathway, including aldehyde dehydrogenase, is well described in the literature. Deletion of these native genes has been described in, e.g., Kozak et al. (2014) Metabolic Engineering 21:46-59). As a result of the attenuation of the native biosynthetic pathway, the flux through the pathway in is reduced by at least 20%, at least 30%, at least 40%, at least 50%, at least 60%, at least 70%, at least 80%, at least 90% or even completely. Methods for attenuating the pathway are described herein and in the literature.

C. Introduction of an Artificial Alternative Pathway for Making Ac-CoA

A second feature of the present compositions and methods is the introduction of an artificial alternative pathway for making Ac-CoA, which artificial pathway does not contribute to a redox cofactor imbalance in the cells under anaerobic conditions. In some embodiments, of the compositions and methods the artificial alternative pathway for making Ac-CoA is the result of introducing exogenous phosphoketolase (EC 4.1.2.22) and exogenous phosphotransacetylase (EC 2.3.1.8) activity to yeast cells. In particular embodiments of the compositions and methods the artificial alternative pathway for making ethanol is the result of introducing a heterologous phosphoketolase gene and a heterologous phosphotransacetylase gene. An exemplary phosphoketolase can be obtained from Gardnerella vaginalis (UniProt/TrEMBL Accession No.: WP_016786789). An exemplary phosphotransacetylase can be obtained from Lactobacillus plantarum (UniProt/TrEMBL Accession No.: WP_003641060).

D. Attenuation of the Native Glycerol Biosynthesis Pathway

Replacing the native biosynthetic pathway for making Ac-CoA with an artificial alternative pathway, as described above, without other genetic manipulation, may be desirable in some instances. However, it is the redox imbalance resulting from attenuation of the native glycerol biosynthesis pathway that causes redox imbalance and provides the impetus for these modifications.

Accordingly, a third feature of the present compositions and methods is attenuation of the native glycerol biosynthesis pathway. Methods for attenuation of the glycerol biosynthesis pathway in yeast are well known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). As a result of the attenuation of the native biosynthetic pathway, the flux through the pathway in is reduced by at least 20%, at least 30%, at least 40%, at least 50%, at least 60%, at least 70%, at least 80%, at least 90% or even completely. Methods for attenuating the pathway are described herein and in the literature.

E. Further Modification to the Yeast

While not critical to the present compositions and methods, the modified yeast may further feature increased acetyl-CoA synthase (also referred to acetyl-CoA ligase) activity (EC 6.2.1.1) to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in ethanol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments of the present compositions and methods, it may be desirable to combine the above-described modifications with the introduction of a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). However, in most embodiments of the present compositions and methods, the introduction of an acetylating acetaldehyde dehydrogenase and/or a pyruvate-formate lyase is not required because the need for these activities is obviated by the attenuation of the native biosynthetic pathway for making Ac-CoA that contributes to redox cofactor imbalance. Accordingly, embodiments of the present compositions and methods expressly lack a heterologous gene(s) encoding an acetylating acetaldehyde dehydrogenase, a pyruvate-formate lyase or both.

In some embodiments, the present modified cells include any number of additional genes of interest encoding protein of interest, such as selectable markers, carbohydrate-processing enzymes, and other commercially-relevant polypeptides, including but not limited to an enzyme selected from the group consisting of a dehydrogenase, a transketolase, a phosphoketolase, a transladolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise modified.

F. Use of the Modified Yeast for Increased Ethanol Production

It is a desired result of the present compositions and methods that the resulting yeast demonstrates increased ethanol production using a carbohydrate substrate compared to a comparable yeast cells lacking the modifications. For example, the modified yeast cells may demonstrate at least 1%, at least 2%, at least 4%, at least 6%, at least 8% or even at least 10% increased ethanol production using a carbohydrate substrate compared to a comparable yeast cells lacking the modifications. It will be understood that the percent increase is relative, such that a 10% increase compared to a normal 15% yield amounts to 16.5% total, not 25%. The modified yeast may also demonstrate altered growth rates or other phenotypes and/or altered production of other valuable biochemicals in addition to ethanol.

G. Yeast for Modification

Yeast is a unicellular eukaryotic microorganisms classified as a member of the fungus kingdom. Yeast that can be used for ethanol production include, but are not limited to, Saccharomyces cerevisiae, other Saccharomyces spp., Kluyveromyces spp. and Schizosaccharomyces spp. Numerous yeast strains are commercially available, many of which have been selected or genetically engineered for desired characteristics, such as high ethanol production, rapid growth rate, and the like. Some yeast has been genetically engineered to produce heterologous enzymes, such as glucoamylase.

While the present compositions and methods are exemplified using a common commercially available strain of Saccharomyces cerevisiae, equivalent modifications can be made and tested in any yeast that include the corresponding native genes for ethanol biosynthesis, including yeast that include further modifications as a result of selection or genetic manipulation, so long as such modifications are not inconsistent with the present compositions and methods.

H. Carbohydrate Substrates and Processes

Ethanol production from a number of carbohydrate substrates is well known, as are innumerable variations and improvements to enzymatic and chemical conditions and mechanical processes. The present compositions and methods are believed to be fully compatible with such substrates and conditions.

### EXAMPLES

### Example 1: Construction of Yeast Strains with Deletions in Ac-CoA and Glycerol Biosynthesis

Three genes that control almost all Ac-CoA production in anaerobically growing yeast (i.e., ALD4, ALD5 and ALD6, which encode major aldehyde dehydrogenases) and two genes that control glycerol production (i.e., GPD1 and GPD2) were deleted from a parental yeast strain using standard molecular biology techniques.

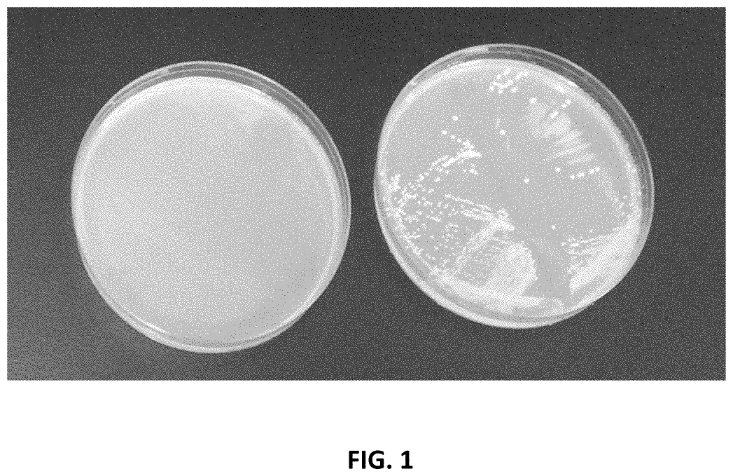

By sequentially deleting each gene, five strains carrying partial or essentially complete disruptions of the Ac-CoA and glycerol biosynthetic pathway were constructed (Table 1). The genotypes were confirmed by amplifying modified regions using PCR and sequencing the resulting PCR products. Although the yeast maybe contain additional endogenous genes encoding aldehyde dehydrogenase homologues, these genes are either expressed at a very low level or the enzymes they encode have very low activity on acetaledyde, as demonstrated by the acetate auxotrophy analysis of strain FGAZ (FIG. 1). Thus, for the purposes described, herein, deletion of ALD4, ALD5 and ALD6 is sufficient to produce a strain with attenuation of the native Ac-CoA biosynthetic pathway.

TABLE 1
Strains with deletions in genes of Ac-CoA
and glycerol biosynthesis

| Strain name | Parent | Genotype |
|---|---|---|
| FG ura3 | FERMAX ™ GOLD | ura3 |
| FG ura3 ald6 | FG ura3 | ura3 ald6 |
| FG ura3 ald5 ald6 | FG ura3 ald6 | ura3 ald5 ald6 |
| FGAZ | FG ura3 ald5 ald6 | ura3 ald4 ald5 ald6 |
| ZG1 | FGAZ | ura3 ald4 ald5 ald6 gpd1 |
| ZZ | ZG1 | ura3 ald4 ald5 ald6 gpd1 gpd2 |

### Example 2. Construction of a Yeast Vector Encoding an Alternative Ac-CoA Biosynthetic Pathway

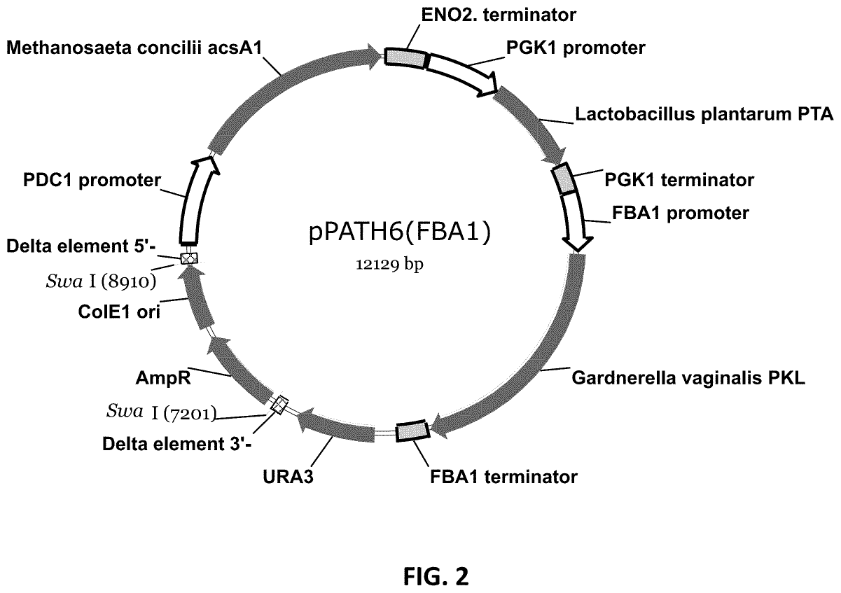

Plasmid vector pPATH6(FBA1) (FIG. 2) was constructed using standard methods. It includes three expression cassettes producing three enzymes of the alternative Ac-CoA biosynthetic pathway: (i) phosphoketolase (EC 4.1.2.22), (ii) phosphotransacetylase (EC 2.3.1.8) and (iii) acetyl-CoA synthetase (EC 6.2.1.1). The synthetic sequences encoding the three enzymes are based on protein sequences of phosphoketolase from Gardnerella vaginalis (WP_016786789), phosphotransacetylase from Lactobacillus plantarum (WP_003641060) and acetyl-CoA synthase from Methanosaeta concilii (WP_013718460). Acetyl-Co synthase, although not a part of phosphoketolase-based Ac-CoA biosynthetic pathway sensu stricto, is used in an auxiliary role to make sure that any acetate produced by chemical or enzymatic hydrolysis of acetyl-phosphate is captured and converted into Ac-CoA.

The coding sequences of the three pathway enzymes in pPATH6(FBA1) was synthesized based on S. cerevisiae codon preferences. These coding sequences were placed under control of strong glycolytic promoters and transcription terminators that were amplified from yeast genomic DNA by PCR. The cluster of three expression cassettes and yeast native ura3 gene (used as selectable marker) were flanked by short stretches of S. cerevisiae sequence to direct DNA integration at any of the multiple δ-sequences present in the yeast genome (FIG. 2). For transformation of yeast, pPATH6(FBA1) was digested with restrictase SwaI to excise sequences required for plasmid maintenance in E. coli. The larger (10.5 kb) fragment from this digest was purified by agarose gel electrophoresis and used to transform strains ZG1 and ZZ (Example 1) to uracil prototrophy. Transformants of strain ZG1 were named as ZG1::pPATH6(FBA1) and transformants of strain ZZ were named as ZZ::pPATH6(FBA1).

### Example 3. Testing the Performance of Strains with Engineered Ac-CoA and Glycerol Pathways

Randomly selected transformants of ZG1 and ZZ, as well as wild-type strain FERMAX™ Gold, were cultivated aerobically overnight in liquid cultures in a medium (SC6%) containing 60 g/l glucose, 1.7 g/l yeast nitrogen base without amino acids and ammonium sulfate, 2 g/l urea. Cells were collected by centrifugation, washed with fresh SC6% medium and used to inoculate cultures in 10 ml of ice-cold SC6% in 20 ml screw-cap vials, to an initial OD₆₀₀ of 0.5. The vials were closed tightly and a 26½ gauge needle was inserted into the cap to provide outlet for the CO₂ generated during fermentation. The fermentations were carried out under strict anaerobic conditions (in an anaerobic hood) at 32° C., with 300 rpm shaking. Samples of the culture broth were taken periodically, sterile-filtered and analyzed by HPLC.

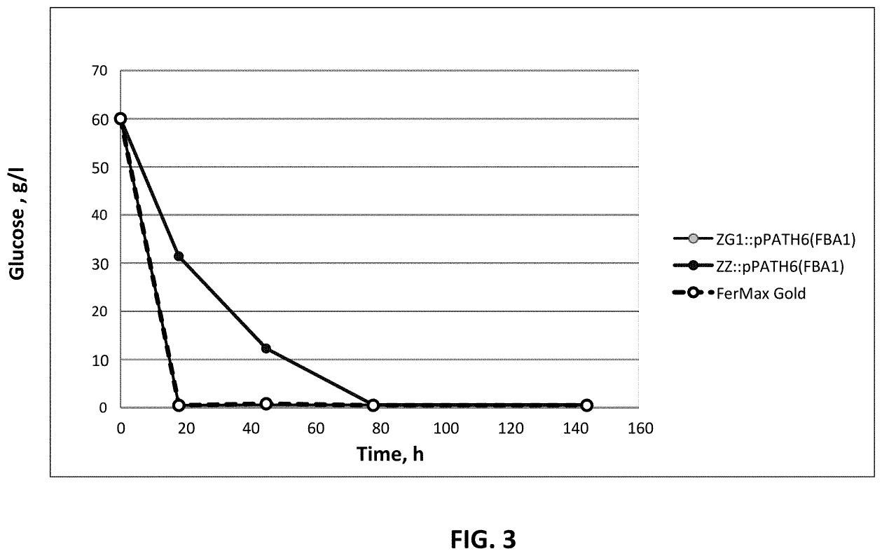

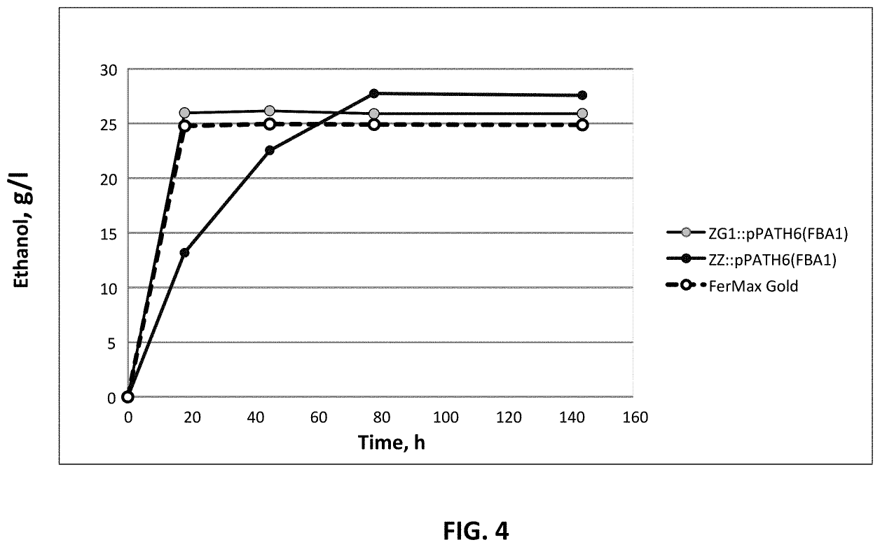

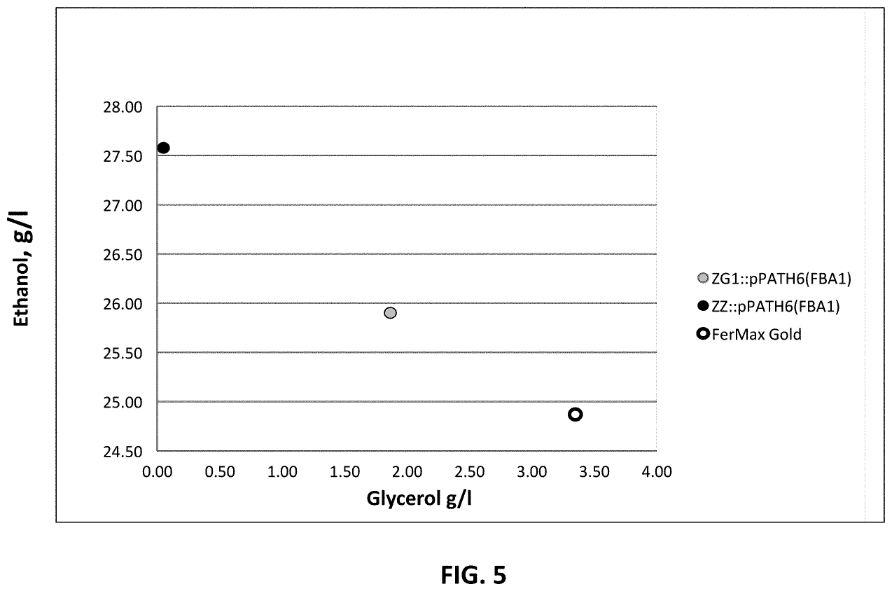

As shown in FIGS. 3 and 4, strain ZG1::pPATH6(FBA1) completely consumed glucose in 18 hours and produced about 5% more ethanol than the wild type strain (i.e., FERMAX™ Gold). Note that the data points and lines for the ZG1::pPATH6(FBA1) data and the FERMAX™ Gold data overlap in FIG. 3. Glycerol production in ZG1::pPATH6(FBA1) was reduced by about 45% relative to wild type (FIG. 5). Fermentation was slower with the ZZ1::pPATH6(FBA1) strain but it is was able to consume all glucose between 45 and 78 hours and produce over 10% more ethanol than the wild type strain (FIGS. 3 and 4). Glycerol production by ZZ1::pPATH6(FBA1) was negligible (FIG. 5).

The results indicated that, in strains with attenuated native Ac-CoA and glycerol pathways, the introduction of an alternative Ac-CoA biosynthetic pathway (pPATH6(FBA1)) not only restores the ability of yeast to grow in the absence of externally added acetate but imparts the ability to produce ethanol in substantially higher yields on glucose compared to wild-type organisms. At the same, glycerol production by such strains is greatly diminished or essentially absent. Wild-type yeast with attenuated glycerol pathways are known to grow poorly and yeast with disrupted glycerol pathways are known to lose the ability to grow anaerobically. Remarkably, despite complete disruption of the glycerol pathway, strain ZZ::pPATH6(FBA1) retains the ability for anaerobic growth and complete conversion of glucose into ethanol with significantly (over 10%) enhanced ethanol yield.

What is claimed is:

1. Modified yeast cells, comprising: an attenuated native biosynthetic pathway for making Ac-CoA, which native pathway contributes to redox cofactor imbalance in the cells under anaerobic conditions; introduction of an artificial alternative pathway for making Ac-CoA, which artificial pathway does not contribute to a redox cofactor imbalance in the cells under anaerobic conditions compared to the native biosynthetic pathway; and attenuation of the glycerol biosynthesis pathway; wherein the modified yeast cells demonstrate increased ethanol production using a carbohydrate substrate compared to a comparable yeast cells lacking the modifications.

2. The modified yeast cells of claim 1, wherein attenuation of the native Ac-CoA pathway is achieved by reducing aldehyde dehydrogenase activity.

3. The modified yeast of claim 1 or 2, wherein attenuation of the native Ac-CoA pathway is achieved by reducing the expression of one or more of the native genes encoding aldehyde dehydrogenase (ALD2, ALD3, ALD4, ALD5 or ALD6).

4. The modified yeast cells of any of claims 1-3, wherein the artificial alternative pathway for making Ac-CoA is the result of introducing exogenous phosphoketolase activity and exogenous phosphotransacetylase activity.

5. The modified yeast cells of any of claims 1-4, wherein the artificial alternative pathway for making Ac-CoA is the result of introducing a heterologous phosphoketolase gene and a heterologous phosphotransacetylase gene.

6. The modified yeast cells of any of claims 1-5, wherein attenuation of the glycerol biosynthesis pathway is the disruption or modification of GDP1, GDP2, GPP1 and/or GPP2.

7. The modified yeast cells of any of the preceding claims, wherein the cells further comprise increased acetyl-CoA synthase activity.

8. The modified yeast cells of any of the preceding claims, wherein the cells further comprise a heterologous gene encoding a polypeptide having acetyl-CoA synthase activity or an overexpressed endogenous gene encoding a polypeptide having acetyl-CoA synthase activity.

9. The modified yeast cells of any of the preceding claims, wherein the cells lack a heterologous gene encoding a protein with NAD⁺-dependent acetylating acetaldehyde dehydrogenase activity or have reduced NAD⁺-dependent acetylating acetaldehyde dehydrogenase activity.

10. The modified yeast cells of any of the preceding claims, wherein the cells lack a heterologous gene encoding a pyruvate-formate lyase or have reduced pyruvate-formate lyase activity.

11. The modified yeast cells of any of the preceding claims, wherein the modified yeast cells demonstrate at least 1%, at least 2%, at least 4%, at least 6%, at least 8% or even at least 10% increased ethanol production using a carbohydrate substrate compared to a comparable yeast cells lacking the modifications.

12. A method for increasing the production of ethanol by yeast cells grown on a carbohydrate substrate, comprising: attenuating the yeast cell native biosynthetic pathway for making Ac-CoA, which native pathway contributes to redox cofactor imbalance in the yeast cells under anaerobic conditions; introducing into the yeast cells an artificial alternative pathway for making Ac-CoA, which artificial pathway does not contribute to a redox cofactor imbalance in the yeast cells under anaerobic conditions compared to the native pathway; and attenuating the glycerol biosynthesis pathway in the yeast cells; wherein the modified yeast cells demonstrate increased ethanol production using a carbohydrate substrate compared to a comparable yeast cells lacking the modifications.

13. The method of claim 12, wherein attenuating the native Ac-CoA pathway is performed by reducing aldehyde dehydrogenase activity.

14. The method of claim 12 or 13, wherein attenuating the native Ac-CoA pathway is performed by disrupting one or more native aldehyde dehydrogenase genes.

15. The method of any of claims 12-14, wherein the artificial alternative pathway for making Ac-CoA results from introducing exogenous phosphoketolase activity and exogenous phosphotransacetylase activity.

16. The method of any of claims 12-15, wherein the artificial alternative pathway for making Ac-CoA is the result of introducing a heterologous phosphoketolase gene and a heterologous phosphotransacetylase gene.

17. The method of any of claims 12-16, wherein attenuating the glycerol biosynthesis pathway is performed by disrupting or modifying GDP1, GDP2, GPP1 and/or GPP2.

18. The method of any of claims 12-17, further comprising increasing acetyl-CoA synthase activity.

19. The method of any of claims 12-18, further comprising introducing into the cell a heterologous gene encoding a polypeptide having acetyl-CoA synthase activity or overexpressing an endogenous gene encoding a polypeptide having acetyl-CoA synthase activity.

20. The method of any of claims 12-19, wherein the cells lack a heterologous gene encoding a protein with NAD⁺-dependent acetylating acetaldehyde dehydrogenase activity or have reduced NAD⁺-dependent acetylating acetaldehyde dehydrogenase activity.

21. The method of any of claims 12-20, wherein the cells lack a heterologous gene encoding a pyruvate-formate lyase or have reduced pyruvate-formate lyase activity.

22. The method of any of claims 12-21, wherein the modified yeast cells demonstrate at least 1%, at least 2%, at least 4%, at least 6%, at least 8% or even at least 10% increased ethanol production using a carbohydrate substrate compared to a comparable yeast cells lacking the modifications.

23. Modified yeast cells produced by the method of any of claims 12-22.

24. A method for increasing the production of ethanol by yeast cells grown on a carbohydrate substrate, comprising: incubating a carbohydrate substrate in the presence of the modified yeast cells of any of claim 1-11 or 23 or in the presence of modified yeast cells produced by the method of any of claims 12-22, wherein the modified yeast cells demonstrate at least 1%, at least 2%, at least 4%, at least 6%, at least 8% or even at least 10% increased ethanol production using a carbohydrate substrate compared to a comparable yeast cells lacking the modifications.

25. Ethanol produced by the method of claim 24 or 28.

26. Modified yeast cells, comprising the acetyl-CoA synthase from Methanosaeta concilii (WP_013718460) or an enzyme having at least 85%, at least 90%, at least 92%, at least 95%, at least 97%, at least 98% and even at least 99% amino acid sequence identity the acetyl-CoA synthase from Methanosaeta concilii.

27. A method for converting acetate to Ac-CoA comprising introducing into the cell a heterologous gene encoding a polypeptide having acetyl-CoA synthase activity, wherein the gene is derived from the acetyl-CoA synthase from Methanosaeta concilii (WP_013718460) or an enzyme having at least 85%, at least 90%, at least 92%, at least 95%, at least 97%, at least 98% and even at least 99% amino acid sequence identity the acetyl-CoA synthase from Methanosaeta concilii.

28. The method of claim 27 used in combination with the method of any of claim 12-22 or 24.
